(19) United States
(12) Patent Application Publication
(10) Pub. No.: US 2019/0231925 A1
(43) Pub. Date: Aug. 1, 2019

(54) Heparin sodium supported hydrogel sustained-release paster

(71) Applicants: CHANGCHUN JA BIOTECH. CO., LTD., Changchun (CN); SHANGHAI PARIN BIO-TECHNOLOGY CO., LTD., Shanghai (CN)

(72) Inventors: Xu HAN, Shanghai (CN); Jingzhe WANG, Changchun (CN); Jing JING, Changchun (CN); Ronghua SUN, Changchun (CN)

(73) Assignees: CHANGCHUN JA BIOTECH. CO., LTD., Changchun (CN); SHANGHAI PARIN BIO-TECHNOLOGY CO., LTD., Shanghai (CN)

(21) Appl. No.: 15/884,414

(22) Filed: Jan. 31, 2018

(51) Int. Cl. A61L 26/00; A61F 13/02

(52) U.S. Cl. CPC A61L 26/0023; A61F 13/0269; A61L 26/0061; A61L 26/008

(57) ABSTRACT

The present invention relates to a medical dressing, in particular to a heparin sodium supported hydrogel sustained-release paster. The inventive heparin sodium supported hydrogel sustained-release paster includes a fixing layer and a backing layer and a hydrogel functional layer successively arranged on the fixing layer. The hydrogel functional layer is made up of hydrophilic high-molecular compound, heparin sodium and aqueous transdermal absorbefacient solution to through high-energy ray radiation or freeze-thaw cycle or combination of high-energy ray radiation and freeze-thaw cycle. The invention has the advantages as follows: the flake solid hydrogel material, synthesized by hydrophilic high-molecular compound with high biosecurity, has features of high biosecurity, high moisture content, compact molecular structure, controllable is water loss rate and prolonged service life. In addition, the hydrogel sustained-release paster is easy in operation, convenient to use, timely to remove in case of discomfortable and adverse reaction and free of deteriorated adverse reaction.

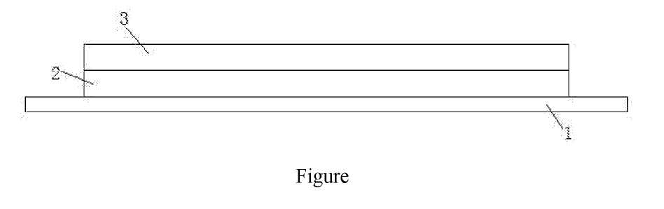

### TECHNICAL FIELD

The present invention relates to a medical dressing, in particular to a hydrogel sustained-release paster supporting heparin sodium.

### BACKGROUND

Heparin sodium is sodium salt of glucosamine sulfate extracted from pulmonary and intestinal mucosa of pigs or bovines, and belongs to mucopolysaccharides substances. It is a kind of anticoagulant, with conventional dosage form comprising injection and cream. The injection, as a conventional dosage form of heparin sodium, results in high local plasma concentration after application, easily causing side effects such as abnormal bleeding in entire body or subcutaneous ecchymosis and induration at injection site; and the cream is easy to become dry with short action duration, incapability to realize long-term drug penetration and poor treatment effect after applied. In summary, the conventional dosage form of heparin sodium exists defects affecting treatment effect in application process.

Hydrogel is a class of polymers with a water-swellable and water-insoluble hydrophilic group and a spatial three-dimensional network structure. The hydrogel material with high biosecurity has features of high water content and tissue biocompatibility, so that it is widely used in fields such as wound care, tissue filler and drug carrier.

Currently, it has been disclosed in patents that the heparin sodium is supported on amorphous hydrogel with Carbomer and alginate as main ingredients. The supported heparin sodium plays treatment effect by cutaneous penetration after applying it in treated area, but has defect that it can not continue to release active ingredients after dehydration drying due to loose molecular structure and rapid moisture loss rate of amorphous hydrogel.

### SUMMARY

The technical problem to be solved by the invention is to provide a hydrogel sustained-release paster supporting heparin sodium for effectively overcoming defects in prior art.

The technical solution for solving above-mentioned technical problem is as follow: a hydrogel sustained-release paster supporting heparin sodium comprises a fixing layer and a backing layer and a hydrogel functional layer successively arranged on the fixing layer. The hydrogel functional layer is made of hydrophilic high-molecular compound, heparin sodium and aqueous transdermal absorbefacient solution through high-energy ray radiation or freeze-thaw cycle or combination of high-energy ray radiation and freeze-thaw cycle.

In the aqueous solution of hydrogel functional layer, the hydrophilic high-molecular compound accounts for 5%-45%, the heparin sodium for 0.15%-3% and the aqueous transdermal absorbefacient solution for 1-5% by weight.

Further, the hydrophilic high-molecular compound is mixture of one or more than two in starch, water-soluble cellulose, water-soluble chitosan, agar, carrageenin, arabic gum, pectin, xanthan gum, carrageenan, alginate, hyaluronic acid, gelatin, bone glue, polyglutamic acid, polylysine, fibrous protein, elastin, keratin, polyacrylamide, polyacrylic acid, polyacrylate, polyvinylpyridine, polyvinylpyrrolidone, polyvinyl alcohol, polymaleic anhydride, polyquaternium, polyethylene glycol, polyethylene oxide or polyvinylpyrrolidone in any proportion.

The beneficial effect of the invention is as follows: the flake solid hydrogel material synthesized by hydrophilic high-molecular compound with high biosecurity has features of high biosecurity, high moisture content, compact molecular structure, controllable water loss rate and prolonged service life. In addition, the hydrogel sustained-release paster made with flake solid hydrogel material as sustained-release ingredient is easy in operation, convenient to use, timely to remove in case of discomfortable and adverse reaction and free of deteriorated adverse reaction.

On the basis of above-mentioned technical solution, the invention also makes the following improvements.

Further, the heparin sodium is bovine-derived heparin sodium or pig-derived low-molecular-weight heparin sodium with content of molecular weight below 16000 accounting for more than 65%.

In addition, the transdermal absorbefacient is laurocapram.

The beneficial effect of above-mentioned technical solution is that the combined lower-molecular-weight heparin sodium and transdermal absorbefacient are supported on hydrogel material and then delivered in sustained release for reducing occurrence of HIT (heparin-induced thrombocytopenia).

Further, the backing layer is semipermeable membrane or nonwoven cloth with water vapor permeability of 500-800 g/(m²·24 h).

The beneficial effect of above-mentioned technical solution is that the backing layer can play a support role for hydrogel layer, strengthen intensity of hydrogel material and avoid hydrogel material from crushing and shedding. Because the backing layer can control loss of hydrogel moisture upwards, it prolongs sustained-release time of medicine to a certain extent.

Further, the fixing layer is of a medical adhesive tape.

The beneficial effect of the above-mentioned fixing layer is to realize tight joint between the hydrogel functional layer and skin through the fixing layer and improve drug delivery effect with simple operation and convenient use.

### BRIEF DESCRIPTION OF THE DRAWINGS

FIGURE is a structural diagram of the inventive hydrogel sustained-release paster supporting heparin sodium.

In the FIGURE, the parts represented by the labels are listed below:

1. fixing layer, 2. backing layer, 3. hydrogel functional layer.

### DETAILED DESCRIPTION OF THE EMBODIMENTS

Next, the inventive principle and features are described with reference to the accompanying FIGURES, and all examples are only used to explain the present invention, not to limit the scope of the present invention.

### Embodiment 1

as shown in the FIGURE, the hydrogel sustained-release paster supporting heparin sodium of the embodiment comprises a fixing layer 1 and a backing layer 2 and a hydrogel functional layer 3 successively arranged on the fixing layer 1, wherein the hydrogel functional layer is made by fully dissolving 10% of polyvinyl alcohol, 0.07% of heparin sodium, 1% of laurocapram and 88.93% of water in weight percent and then freeze-thaw cyclic synthesis; the backing layer is semipermeable membrane with water vapor permeability of 500 g/(m²·24 h), and the backing layer 2 is tightly bonded with the hydrogel functional layer 3; the fixing layer 1 is of a medical adhesive tape bonded with the backing layer 2 tightly.

About 12 hours after applying the hydrogel sustained-release paster on abdominal area, the result, detected by heparin sodium content in hydrogel functional layer 3 and four coagulation tests, shows 44% of human's absorbing capacity to heparin sodium and human's coagulation four indices similar to low-molecular-weight heparin sodium absorbed by human body.

### Embodiment 2

the hydrogel sustained-release paster supporting heparin sodium of the embodiment comprises a fixing layer 1 and a backing layer 2 and a hydrogel functional layer 3 successively arranged on the fixing layer 1, wherein the hydrogel functional layer is made by fully dissolving 10% of polyvinyl alcohol, 2% of polyvinylpyrrolidone, 1% of heparin sodium, 5% of laurocapram and 82% of water in weight percent and then high-energy electron beam radiation synthesis; the backing layer is nonwoven cloth with water vapor permeability of 800 g/(m²·24 h), and the backing layer 2 is tightly bonded with the hydrogel functional layer 3; the fixing layer 1 is of a medical adhesive tape, separated from the sustained-release paster, by which the hydrogel sustained-release paster is fixed on wounded parts during use.

About 24 hours after applying the hydrogel sustained-release paster on abdominal area, the result, detected by heparin sodium content in hydrogel functional layer 3 and four coagulation tests, shows 60% of human's absorbing capacity to heparin sodium and human's coagulation four indices similar to low-molecular-weight heparin sodium absorbed by human body.

### Embodiment 3

the hydrogel sustained-release paster supporting heparin sodium of the embodiment comprises a fixing layer 1 and a backing layer 2 and a hydrogel functional layer 3 successively arranged on the fixing layer 1, wherein the hydrogel functional layer is made by fully dissolving 20% of polyacrylic acid, 25% of starch, 3% of heparin sodium, 2% of laurocapram and 50% of water in weight percent and then gamma radiation synthesis; the backing layer is nonwoven cloth with water vapor permeability of 650 g/(m²·24 h), and the backing layer 2 is tightly bonded with the hydrogel functional layer 3; the fixing layer 1 is of a medical adhesive tape bonded with the backing layer 2 tightly.

About 24 hours after applying the hydrogel sustained-release paster on abdominal area, the result, detected by heparin sodium content in hydrogel functional layer 3 and four coagulation tests, shows 66% of human's absorbing capacity to heparin sodium and human's coagulation four indices similar to low-molecular-weight heparin sodium absorbed by human body.

### Embodiment 4

the hydrogel sustained-release paster supporting heparin sodium of the embodiment comprises a fixing layer 1 and a backing layer 2 and a hydrogel functional layer 3 successively arranged on the fixing layer 1, wherein the hydrogel functional layer is made by fully dissolving 10% of polyvinyl alcohol, 2% of polyvinylpyrrolidone, 1% of xanthan gum, 0.5% of heparin sodium, 2.5% of laurocapram and 84% of water in weight percent and then combination of freeze-thaw cyclic synthesis and high-energy electron beam radiation synthesis; the backing layer is semipermeable membrane with water vapor permeability of 700 g/(m²·24 h), and the backing layer 2 is tightly bonded with the hydrogel functional layer 3; the fixing layer 1 is of a medical adhesive tape bonded with the backing layer 2 tightly.

About 24 hours after applying the hydrogel sustained-release paster on human calves, the result, detected by heparin sodium content in hydrogel functional layer 3 and four coagulation tests, shows 29% of human's absorbing capacity to heparin sodium and human's coagulation four indices similar to low-molecular-weight heparin sodium absorbed by human body.

Embodiment 5: the hydrogel sustained-release paster supporting heparin sodium of the embodiment comprises a fixing layer 1 and a backing layer 2 and a hydrogel functional layer 3 successively arranged on the fixing layer 1, wherein the hydrogel functional layer is made by fully dissolving 2% of sodium polyacrylate, 5% of polyethylene glycol, 5% of polyving alcohol, 1% of heparin sodium, 1% of laurocapram and 86% of water in weight percent and then freeze-thaw cyclic synthesis; the backing layer is nonwoven cloth with water vapor permeability of 700 g/(m²·24 h), and the backing layer 2 is tightly bonded with the hydrogel functional layer 3; the fixing layer 1 is of a medical adhesive tape bonded with the backing layer 2 tightly.

About 24 hours after applying the hydrogel sustained-release paster on abdominal area, the result, detected by heparin sodium content in hydrogel functional layer 3 and four coagulation tests, shows 50% of human's absorbing capacity to heparin sodium and human's coagulation four indices similar to low-molecular-weight heparin sodium absorbed by human body.

In above-mentioned five embodiments, the backing layer 2 is connected with the fixing layer 1 by a bonding mode, or a fixed or a detachable mode for convenient use and storage.

The above statements are just preferred embodiments of the present invention rather than limitation. Any modification, equal to replacement and improvement within the spirit and principle of the invention, should be included in protective range of the invention.

What is claimed is:

1. A hydrogel sustained-release paster supporting heparin sodium, comprising a fixing layer, a backing layer and a hydrogel functional layer; wherein the backing layer and the hydrogel functional layer are successively arranged on the fixing layer; the hydrogel functional layer is made of an aqueous solution comprising a hydrophilic compound, the heparin sodium and an aqueous transdermal absorbefacient through high-energy ray radiation or freeze-thaw cycle or combination of high-energy ray radiation and freeze-thaw cycle.

2. The hydrogel sustained-release paster supporting the heparin sodium according to claim 1, wherein the aqueous solution of the hydrogel functional layer comprises the 5%-45% hydrophilic compound by mass, the 0.07%-5% heparin sodium by mass and the 1-5% aqueous transdermal absorbefacient by mass.

3. The hydrogel sustained-release paster supporting the heparin sodium according to claim 2, wherein the hydrophilic compound is one or more selected from the group consisting of starch, water-soluble cellulose, water-soluble chitosan, agar, carrageenin, arabic gum, pectin, xanthan gum, carrageenan, alginate, hyaluronic acid, gelatin, bone glue, polyglutamic acid, polylysine, fibrous protein, elastin, keratin, polyacrylamide, polyacrylic acid, polyacrylate, polyvinylpyridine, polyvinylpyrrolidone, polyvinyl alcohol, polymaleic anhydride, polyquarternium, polyethylene glycol, polyethylene oxide and polyvinylpyrrolidone in any proportion.

4. The hydrogel sustained-release paster supporting the heparin sodium according to claim 2, wherein the heparin sodium is a bovine-derived heparin sodium or a pig-derived low-molecular heparin sodium, wherein a content of the heparin sodium with a molecular weight of less than 16000 is more than 65%.

5. The hydrogel sustained-release paster supporting the heparin sodium according to claim 2, wherein the transdermal absorbefacient is a laurocapram.

6. The hydrogel sustained-release paster supporting the heparin sodium according to claim 1, wherein the backing layer is a semipermeable membrane or a nonwoven cloth with a water vapor permeability of 500-800 g/(m²·24 h).

7. The hydrogel sustained-release paster supporting the heparin sodium according to claim 1, wherein the fixing layer is a medical adhesive tape.

8. The hydrogel sustained-release paster supporting the heparin sodium according to claim 2, wherein the backing layer is a semipermeable membrane or a nonwoven cloth with a water vapor permeability of 500-800 g/(m²·24 h).

9. The hydrogel sustained-release paster supporting the heparin sodium according to claim 3, wherein the backing layer is a semipermeable membrane or a nonwoven cloth with a water vapor permeability of 500-800 g/(m²·24 h).

10. The hydrogel sustained-release paster supporting the heparin sodium according to claim 4, wherein the backing layer is a semipermeable membrane or a nonwoven cloth with a water vapor permeability of 500-800 g/(m²·24 h).

11. The hydrogel sustained-release paster supporting the heparin sodium according to claim 5, wherein the backing layer is a semipermeable membrane or a nonwoven cloth with a water vapor permeability of 500-800 g/(m²·24 h).

12. The hydrogel sustained-release paster supporting the heparin sodium according to claim 2, wherein the fixing layer is a medical adhesive tape.

13. The hydrogel sustained-release paster supporting the heparin sodium according to claim 3, wherein the fixing layer is a medical adhesive tape.

14. The hydrogel sustained-release paster supporting the heparin sodium according to claim 4, wherein the fixing layer is a medical adhesive tape.

15. The hydrogel sustained-release paster supporting the heparin sodium according to claim 5, wherein the fixing layer is a medical adhesive tape.
